(19) United States
(12) Patent Application Publication
(10) Pub. No.: US 2020/0327616 A1
(43) Pub. Date: Oct. 15, 2020

(54) PARTIALLY PRIVATE AND VERIFIABLE DATA EXCHANGE

(71) Applicant: Blockadoc, LLC, Carmel, IN (US)

(72) Inventors: Anthony J. Burke, Noblesville, IN (US); Jeffrey S. Goens, Carmel, IN (US); Bruce M. Goens, Zionsville, IN (US)

(73) Assignee: Blockadoc, LLC, Carmel, IN (US)

(21) Appl. No.: 16/845,729

(22) Filed: Apr. 10, 2020

Related U.S. Application Data
(60) Provisional application No. 62/832,240, filed on Apr. 10, 2019.

(51) Int. Cl. G06Q 40/08; G16H 80/00; H04L 9/06

(52) U.S. Cl. CPC G06Q 40/08; G16H 80/00; G06Q 2220/00; H04L 2209/38; H04L 2209/56; H04L 9/0643

(57) ABSTRACT

A transactional medical data exchange provides a combination of public and private communication channels through which parties to a transaction, such as a medical care provider and a health insurance provider, may exchange information. When such an exchange is needed, the parties create records on the public communication channel that serve as a publicly viewable record that communication has begun. Information from these public records are used to share messages, along with messages, files, and other information related to care, via a private communication channel. Each private communication is sent and stored with unique authenticating data to form a chain of verifiable transactions. When communication is done, each party creates records on the public communication that request and acknowledge that communication is now complete and provide publicly viewable information that can be used to verify and authenticate each private message by itself, or in combination with others.

### CROSS-REFERENCE TO RELATED APPLICATIONS

This application claims priority to U.S. Provisional Patent Application 62/832,240, filed Apr. 10, 2019; titled “PARTIALLY PRIVATE AND VERIFIABLE DATA EXCHANGE”; and is hereby incorporated by reference as if set forth in its entirety.

### FIELD

The disclosed technology pertains to a system for a multi-channel data exchange.

### BACKGROUND

Many industries rely upon timely and accurate exchanges of information between multiple parties. When an error occurs, whether caused by technology or a business process, and data is not timely transmitted, received, or acted upon, a business may not receive services or goods in time to use them, may have payment for a good or service delayed or canceled entirely, or may otherwise be impacted in a way that reduces their ability to service their customers. In some cases, the result of such an error may be relatively trivial, such as where a retail store temporarily has low stock of paper towels or bananas due to errant data. However, in other cases, such as in healthcare, a miscommunication of data can lead to serious harm, including delays in treatment for a patient, financial harm to a patient or provider due to denial of insurance coverage, or improper treatment for a patient.

Miscommunication or lack of communication of data is a particular problem in health care, due in part to the variety of providers and insurers that may be involved in any single patient event. As an example, a patient suffering from back pain may receive care from a primary care medical practice, a radiology practice, and an orthopedic practice. Each of these providers relies upon information from the others, and the aggregate information from the care may be needed by one or more health insurance carriers or other payers in order to evaluate the event and determine what portion of the care is covered by the insurer or other payer, and, in some cases, which types of care are authorized or allowed to be provided to the patient under their insurance or other benefit program.

In some cases, an intermediary, such as a health information exchange, may operate as a data pass-through for the many parties involved in providing care in the above scenario. The intermediary may provide communication channels and data repositories though which data may be transmitted, stored, and accessed by other authorized parties.

Such intermediaries may improve the overall ability to communicate between parties, but still have shortcomings. As an example, a particular intermediary may receive and store information from a health care provider that is requesting payment for a particular patient event, but the insurance provider responsible for payment may be having technical issues that prevent them from receiving notice of the information.

As another example, in some cases, intermediaries may not provide the technical systems and associated services that might allow a health care provider, insurance provider or other payer, or other party to review, audit, or otherwise verify the exchange of information through the intermediary to determine how and why a miscommunication might occur. Especially where health data is concerned, it may be difficult or impossible for a party to gather the information needed from such a closed system in order to prove that a payment is appropriate or unnecessary, or to prove which party may be responsible for a miscommunication and should bear the costs of that miscommunication.

What is needed, therefore, is an improved system for exchanging data.

### BRIEF DESCRIPTION OF THE DRAWINGS

The drawings and detailed description that follow are intended to be merely illustrative and are not intended to limit the scope of the invention as contemplated by the inventor(s).

FIG. 1 shows a schematic diagram of an exemplary architecture for a multi-provider, multi-payer medical data exchange;

FIG. 2 shows a schematic diagram of an exemplary architecture for a multi-provider, multi-payer medical data exchange with an intermediary;

FIG. 3A shows a schematic diagram of an exemplary architecture for a transactional data exchange with an intermediary;

FIG. 3B shows a schematic diagram of an exemplary architecture for a transactional data exchange with no intermediary;

FIG. 4 shows a schematic diagram of an exemplary participant in the transactional data exchange of FIGS. 3A or 3B;

FIG. 5 shows a flowchart of a set of high-level steps that could be performed to provide transactional data exchange using the systems of FIGS. 3A or 3B;

FIG. 6 shows a flowchart of an exemplary set of steps that could be performed to open a transactional exchange channel;

FIG. 7 shows a flowchart of an exemplary set of steps that could be performed to exchange messages over the transactional exchange channel;

FIG. 8 shows a flowchart of an exemplary set of steps that could be performed to close the transactional exchange channel;

FIG. 9 shows a schematic diagram that illustrates aspects of FIGS. 5-8;

FIG. 10 shows a schematic diagram that illustrates an exemplary transaction chain that may be created by performing steps such as those in FIGS. 5-8; and

FIG. 11 shows a schematic diagram that illustrates exemplary data associated with the transaction chain of FIG. 10.

### DETAILED DESCRIPTION

The inventors have conceived of novel technology that, for the purpose of illustration, is disclosed herein as applied in the context of a system for multi-channel data exchange. While the disclosed applications of the inventors' technology satisfy a long-felt but unmet need in the art of multi-channel data exchanges, it should be understood that the inventors' technology is not limited to being implemented in the precise manners set forth herein but could be implemented in other manners without undue experimentation by those of ordinary skill in the art in light of this disclosure. Accordingly, the examples set forth herein should be understood as being illustrative only and should not be treated as limiting.

Turning now to the figures, FIG. 1 shows a schematic diagram of an exemplary architecture for a multi-provider, multi-payer medical data exchange (10). In the data exchange (10), a provider (12) and a provider (20) are separate health care providers (e.g., a general health care provider, and an orthopedic care provider). Each provider (12, 20) sees patients having a variety of health insurance providers, and so exchange information with a number of payers (14, 16, 18). Each provider (12, 20) is in communication with each payer (14, 16, 18), as well as in communication with each other. Communication may include telephone calls, email exchanges, paper invoicing and payment, or communication through proprietary software interfaces, web portals, and other software applications. As can be seen, a provider (12) must manage communications with multiple other parties (e.g., the payers (14, 16, 18) and the other provider (20)), which each may provide distinct requirements for the exchange of data. As an example, the payer (14) may require paper invoicing from provider (12), while the payer (16) may require that the provider (12) submit information via a proprietary web portal, and the payer (18) may require that the provider (12) submit information directly from their own information system using a software application programming interface, or API.

With such requirements, it can be seen that provider (12) has a myriad of requirements in order to request and receive payments from payers (14, 16, 18), and to exchange information from provider (20) when they provide related care to the same patient (e.g., where provider (20) may have to provide x-ray images and analyses to provider (12) prior to a follow up appointment with the patient). In such a system, it is likely—if not inevitable—that invoices and payments will be missed, lost, or delayed, and that patient data will not be available between providers when it ideally should be.

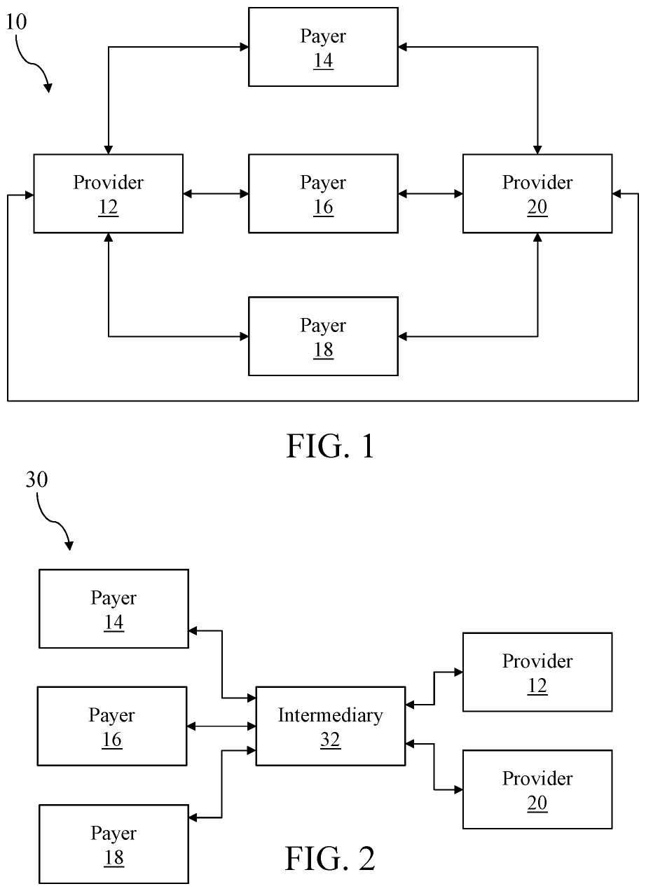

As has been described, in some cases an intermediary (32) may be available to facilitate communication between payers and providers, such as is shown in FIG. 2, which shows a schematic diagram of an exemplary architecture for a multi-provider, multi-payer medical data exchange (30) with an intermediary. The intermediary (32) may be a third party such as a health information exchange. The intermediary (32) can provide benefits such as standardized record formatting, record maintenance, standardized communication interfaces, and delivery or notification of the availability of new data related to a patient or payment. With the medical data exchange (30), the intermediary (32) can serve as a file and data repository that each payer (14, 16, 18), and each provider (12, 20) can communicate with using standardized data formatting and interfaces (e.g., the intermediary (32) may provide a web portal or software service that runs on an individual payer's pre-existing information system). In this manner, the provider (12) only needs to support communication with the intermediary (32), rather than communication with each of the payers (14, 16, 18) and the provider (20) as in FIG. 1.

While the medical data exchange (30) provides some advantages over the medical data exchange (10), it is very dependent upon the intermediary (32). As a result, technical shortcomings in the intermediary (32) systems can result to a complete loss of communication between payers and providers. Such a system also allows the intermediary (32) to limit access to the data, records, and history of data exchanged through the intermediary (32) on their own terms, with little recourse for the provider (12) or the payer (14) in the event of a dispute. As an example, if the provider (12) has a dispute with the payer (14) over whether a particular request for payment was submitted within a proper time frame or with a proper form, the provider (12) may be dependent upon the intermediary (32) to provide additional information confirming that the request for payment was properly received, and whether or not the payer (14) received a notification of the request, or ever accessed the request.

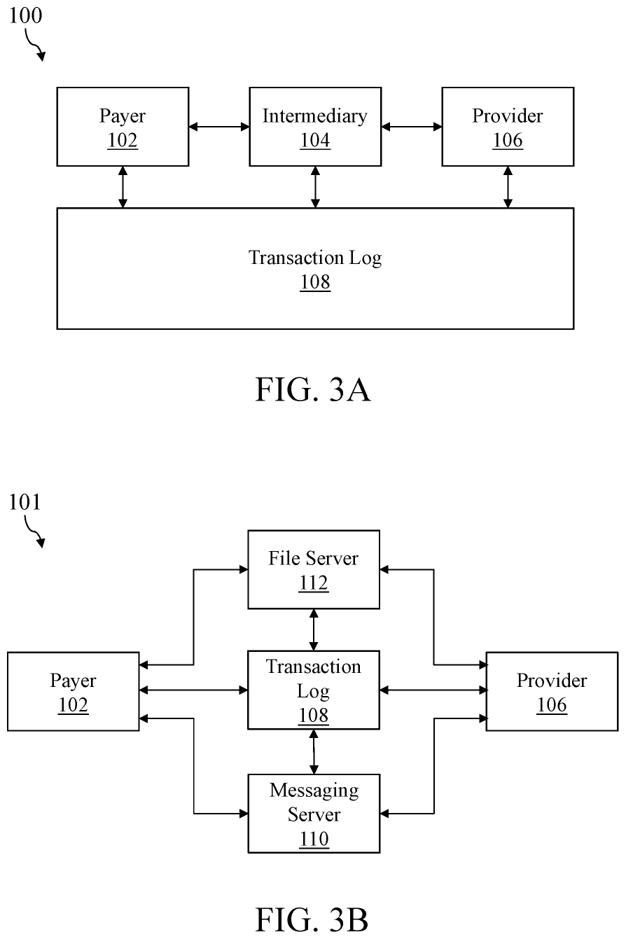

In order to provide a more robust data exchange, and to improve the level of accountability for each party, a system such as that shown FIG. 3A may be implemented. FIG. 3A shows a schematic diagram of an exemplary architecture for a transactional data exchange (100). In the transactional data exchange (100), each of a payer (102) and a provider (106) communicates with the intermediary (104) as in FIG. 2. A transaction log (108) is also accessible to each of the payer (102), the intermediary (104), and the provider (106) and provides a separate communication channel that builds onto the communication channel provided by the intermediary (104).

As an example, communication with the intermediary (104) may be peer-to-peer using a software API or other direct interface, and it may be a private communication channel on which communications may only be accessible to the involved parties. The transaction log (108) may instead be a more public communication channel, such as a more widely accessible blockchain structure or other distributed ledger, on which the communications may be accessible or viewable to many parties, or even to any party in the world. In such an example, records or entries created on the transaction log (108) during communication between the parties will be publicly viewable and verifiable, as will be described in more detail below, and thus will provide any party (e.g., the payer (102), the provider (106), or a third-party auditor or reviewer) the ability to examine and verify communications without being entirely dependent upon the intermediary (32) to provide access to such data.

In order to provide end-to-end accountability and verification of transactions, communications with the intermediary (104) will enforce transactional aspects, such as requiring each party that receives a message to actively acknowledge receipt of a message before it is treated as received, and will also preserve unique data from message to message in order to form a chain of messages whose content can be verified individually from anywhere within the chain, without requiring the full contents of the chain to be provided to the verifying party, as will be explained in more detail below.

FIG. 3B is a schematic diagram of an exemplary architecture for a transactional data exchange (101) with no intermediary, which may function similarly to the transactional data exchange (100) as described above. As can be seen, each of the payer (102) and the provider (106) communicates directly with the transaction log (108) (e.g., in order to create publicly viewable records or entries that indicate the creation and termination of a communication channel as described herein). Instead of the intermediary (104) however, each party communicates directly with a messaging server (110) and a file server (112) in order to exchange messaging and files. In such an implementation, separate parties could operate the file server (112) and the messaging server (110) in order to split these functions out, which may provide further advantages in terms of anonymizing and securing data. For example, the messaging server (110) may provide the private communication channel between parties and may hold minimal or no patient health information, while the file server (112) may be the primary or sole repository of such information. In such an implementation, an entity such as the intermediary (104) may provide robust security and maintenance of records on the file server (112), while another party provides encrypted or anonymized private messaging via the messaging server (110), or vice versa.

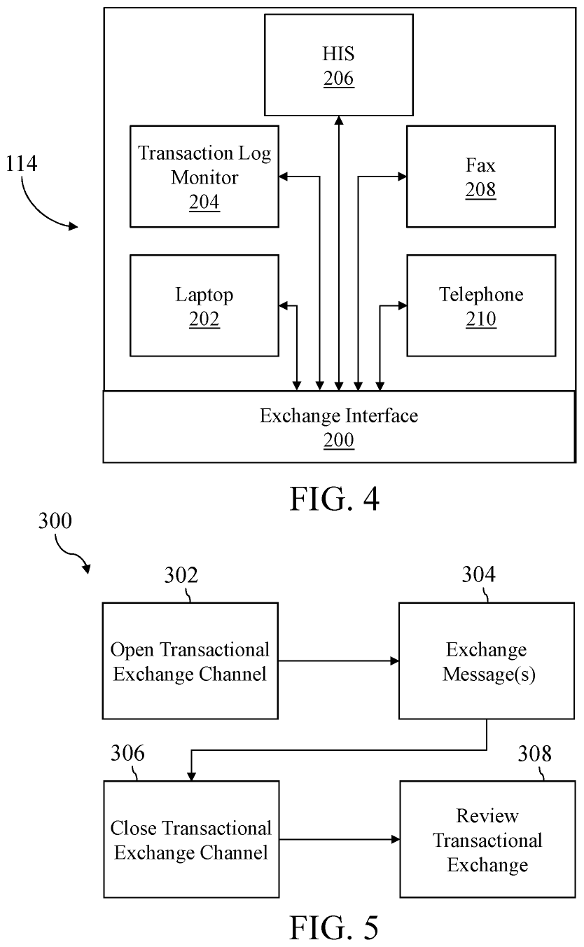

FIG. 4 is a schematic diagram of an exemplary participant in the transactional data exchange of FIGS. 3A or 3B. With a system such as the disclosed transactional data exchanges (100, 101), it may be advantageous to have all communication between parties be captured (e.g., by the intermediary (104) or the messaging and file servers (110, 112). This can be primarily accomplished by providing highly usable and flexible software interfaces, but there are still additional types of communication, such as phone calls and faxes, that will not be captured using conventional information systems. Additionally, with reference to FIG. 3B in particular, it may be disadvantageous to provide a platform where the payer (102) must support communications with the transaction log (108), the messaging server (110), and the file server (112). To address some of these concerns, some participants in transactional data exchanges (100, 101) may be configured as shown in FIG. 4. A participant (114), which may be a care provider, a payer, or another party to transactions, may communicate with the transactional data exchange (100, 101) via an exchange interface (200). The exchange interface (200) may be a software process and interface configured to monitor incoming and outgoing communications for the participant (114) and capture desired communications so that they can be either completely transmitted or at least partially mirrored or registered via the private communication channel.

As an example, this may include the exchange interface (200) being configured to monitor communication from devices such as laptops (202), which may include email, messaging, and interactions through web portals or other software applications. This may also include interactions with a hospital information system, or “HIS” (206), from which patient records, care events, diagnosis, and other information may be transmitted and received. This may also include fax machines (208), scanners, and other similar devices which may be used to capture and transmit documents. This may also include telephone (210) conversations, either by telephone (e.g., a landline or VOIP telephone that is routed through the exchange interface (200)) or mobile devices (e.g., a mobile phone that uses a software application to facilitate phone calls between parties, or to submit information from such phone calls via the exchange interface (200)), between parties to the transaction. With any such communications passing at least partially through the exchange interface (200), their occurrence (e.g., the date, time, parties involved, a unique identifier, etc.) if not their entire contents (e.g., the actual fax image, the audio of a phone conversations) may be transmitted and acknowledged through the private messaging channel.

As an example, where a telephone conversation occurs between parties over the telephone (210) that is passed through the exchange interface (200), a message may be transmitted via the private communication channel describing the date and time, the numbers of the caller and the recipient, a unique identifier associated with the call that anonymously identifies a patient or care event, and may also contain an audio recording or a speech-to-text transcript of the conversation. Even where the telephone in use is not passed through the exchange interface (200), such information may still be manually provided by participants to the call during or after the call (e.g., such information may be entered via web portal or other software interface, along with notes from and/or transcription of the call).

The participant (114) in a transactional data exchange (100, 101) also includes a transaction log monitor (204), which may be a software process that runs on a server (e.g., a standalone server, or on a pre-existing server such as the HIS (206)). The transaction log monitor (204) may be configured to run continuously and to monitor the transaction log (108) for records or entries that are of interest to the participant (114). For example, where the participant (114) is the provider (106), the transaction log monitor (204) may constantly search the transaction log (108) for new publicly viewable entries that are associated with a unique identifier for a patient, patient event, or ongoing care that is being provided by the provider (106). Changes to the transaction log (108) that are of interest may include, for example, public messages indicating that the payer (102) has requested that a communication channel be opened over the transactional data exchange (100), that the payer (102) has transmitted a private message that has not yet been acknowledged by the provider (106), or that the payer (102) has requested that the communication channel be closed, as will be described in more detail below.

FIG. 5 is a flowchart of a set of high-level steps (300) that could be performed to provide transactional data exchange using the systems of FIGS. 3A or 3B. When communication is needed, such as when a patient enters a health care provider's facility and requests medical care for some condition, a transactional exchange channel may be opened (302) to allow the parties to reliably and verifiably communicate throughout the care of that patient. Opening (302) the transactional exchange channel may include creating publicly viewable records on the transaction log (108) indicating the start of communication and linking the publicly viewable records to a chain of messages exchanged over the private channel (e.g., through the intermediary (104)). Once opened, one or more messages may be exchanged (304) through the transactional exchange channel throughout the care of the patient, with message exchanges being acknowledged when received, and including unique linking data that allows one or more of the messages to be verified, as will be described in more detail below.

When the parties are ready to close the channel for any reason (e.g., the status of the patient (e.g., “in-patient” or “observation”) is determined, the course of treatment for the patient ends, and/or the parties have resolved any open issues, disputes, payments, or other concerns), the transactional exchange channel may be closed (306), which may include creating publicly viewable records on the transaction log (108) indicating completion of the transaction, and may contain data, such as the Merkle root of the conversation, which could allow any one or more messages of the transaction to be verified independently of the content of the other messages. With records associated with opening (302) and closing (306) the exchange being publicly recorded and immutable, any party may review (308) some or all of the exchange, for example, to verify completion of the exchange, to audit compliance with the protocol by each participant (114), or to verify receipt or content of individual messages transferred over the exchange. As an example, this may include calculating and verifying a chain of cryptographic hashes associated with each message, where the hash associated with each message is sequentially built upon previous messages or their hashes. Thus, with a list of hashes for each message of the chain, one or more individual messages can be verified by calculating and verifying their hashes forward in the sequence through the final hash code, which may be associated with the public records indicating closing (306) of the exchange channel. In this manner, any party can verify each message exchange, without needing to review (or even have access to) the entire content of the messages in that exchange, which may include patient health information or other sensitive private information.

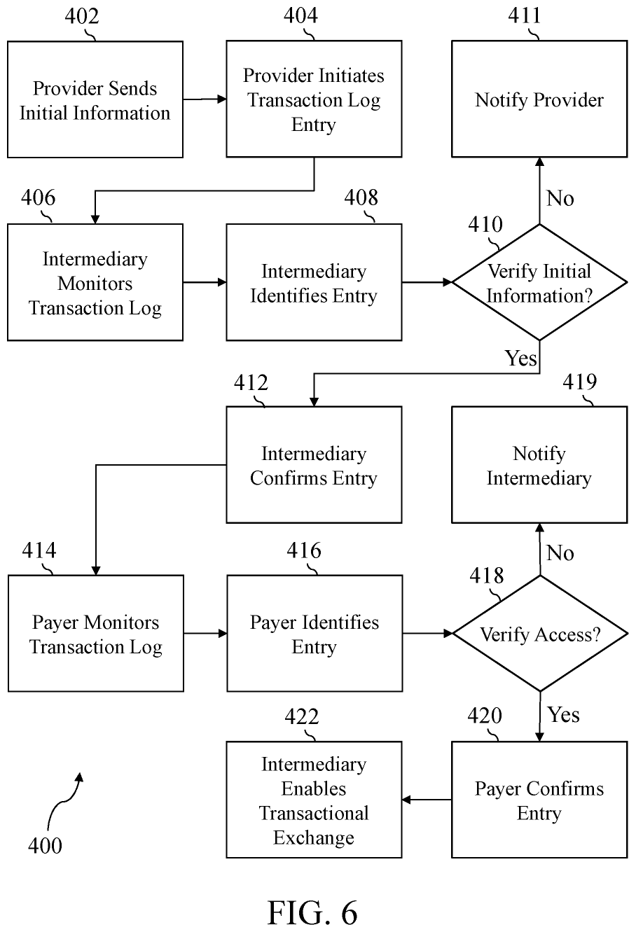

FIGS. 6-8 provide additional examples related to opening an exchange channel, communicating over an exchange channel, and closing an exchange channel, respectively in a transactional data exchange (100, 101). FIG. 6 is a flowchart of an exemplary set of steps (400) that could be performed to open a transactional exchange channel. In some implementations, these steps (400) may be performed in parallel with a patient arriving at a care provider (e.g., such as the provider (106)) and requesting treatment. The provider (106) may send a set of initial information to the intermediary (104) using a private communication channel (e.g., a software interface, web portal, or other private interface) that may include initial patient information, such as name, age, medical condition reported, and other similar information. The provider (106) may also initiate (404) an entry to the transaction log (108) indicating the provider's (106) recognition that a communication channel is needed.

As an example, this may include creating a record on a public blockchain or other public ledger or database that includes a patient identifier (e.g., a random or unique anonymous identifier), identifiers for the payer (102), the provider (106), and the intermediary (104) that are involved in the exchange (e.g., for each, a random or unique identifier), and unique hash information based upon the provided (402) initial information. The unique hash information may be, for example, a hash of each message, file, or other information provided (402) in the initial information in a Merkle-tree structure. The unique hash information may also include unique hash information associated with the block, ledger entry, or other database record to which the initiated (404) entry was written. For example, the unique information could include a unique hash built from the initial information and a separate unique hash associated with that block, ledger entry, or record, or could include a single unique hash built from the combination of the two. When used herein, a channel open request should be understood to include one or more of the provided (402) initial information, information used to initiate (404) the transaction log entry, or other similar information, or combinations thereof, while an open request record should be understood to include one or more of the created transaction log entry (404) or other similar information, or combinations thereof.

The intermediary (104) may monitor (406) the transaction log (108), using a process such as the transaction log monitor (204), for changes that are associated with the intermediary (104), and it may identify (408) the initiating (404) entry being added to the transaction log (108) based upon the inclusion of information uniquely identifying the intermediary (e.g., a unique intermediary identifier, string value, or signature using a cryptographic key associated with the intermediary (104)). Once such a change is identified (408), the intermediary (104) may verify (410) the initial information, which may include comparing a hash of the provided (402) information, which may include electronic messages, files, and other information, to the unique hash values associated with the initiation (404) entry to verify that every file was received and is available to the intermediary (104). If files cannot be verified, the intermediary (104) may notify (411) the provider (106) to indicate that there is a mismatch between the initial information and the hashed verifier for that information. In such a case, the provider (106) may re-send or re-attempt opening the transactional data exchange by returning to the step of sending (402) the initial information. In some implementations, this exchange regarding the mismatch is documented in the transaction log (108), while in others the intermediary (104) simply withholds its acknowledgment of the initiating (404) entry until the mismatch is resolved. Where the initial information can be verified, the intermediary (104) confirms (412) the initiation of the transaction log entry by writing a records to the blockchain, public ledger, or other database to indicate the intermediary's (104) recognition of the communication channel request. When used herein, an open confirmation record should be understood to include one or more of the confirming (412) record or other confirmation information that is created on the database or otherwise provided to a recipient in order to indicate a confirmed request to communicate.

The payer (102) may monitor (414) the transaction log, using a process such as the transaction log monitor (204), for changes that are associated with the payer (102) and may identify (416) the initiating (404) entry being added to the transaction log (108) based upon the inclusion of information uniquely identifying the payer (e.g., a unique payer identifier, string value, or signature using a cryptographic key associated with the payer (102)). When used herein, a response identifier should be understood to include information uniquely identifying the payer or another recipient of a request to communicate or may include information that is otherwise usable to monitor (414) the transaction log for such requests. Once identified (416), the payer (102) may verify (419) that the initial information can be accessed (e.g., which may include accessing electronic messages, files, or other data stored by the intermediary (104), or stored in the file server (112), for example) by comparing the available data to the associated hash values. Where the information cannot be accessed, the payer (102) may notify (419) the intermediary (104) to indicate a mismatch, which the intermediary (104) may address by restoring or updating files, or by requesting that the provider (106) return to the step of sending (402) the initial information. In some implementations, this exchange regarding the mismatch is documented in the transaction log (108), while in others the payer (102) simply withholds its confirmation of the initiating (404) entry until the mismatch is resolved.

Where access to and correctness of the initial information can be verified, the payer (102) may confirm (420) the initiating entry by writing a record to the blockchain, public ledger, or other database indicating that the payer is confirming opening of the transactional data exchange and has access to the initial information. When used herein, a response confirmation record should be understood to include one or more of the payer confirmation (420) record or other similar information that indicates a recipient's confirmed desire to communicate. When available, a response confirmation record may be identified by a system or party such as the intermediary (104) by querying the database. With each of the parties having publicly acknowledged the details around the creation of the transactional data exchange, the intermediary (104) (e.g., or the messaging server (110)) may enable (422) ongoing transactional exchange of messages over the private channel by providing each of the payer (102) and the provider (106) authorization and/or cryptographic keys necessary to transmit messages through the private channel, which may include electronic messages, files, and other data, via the private channel, without requiring additional creation of public records for each message.

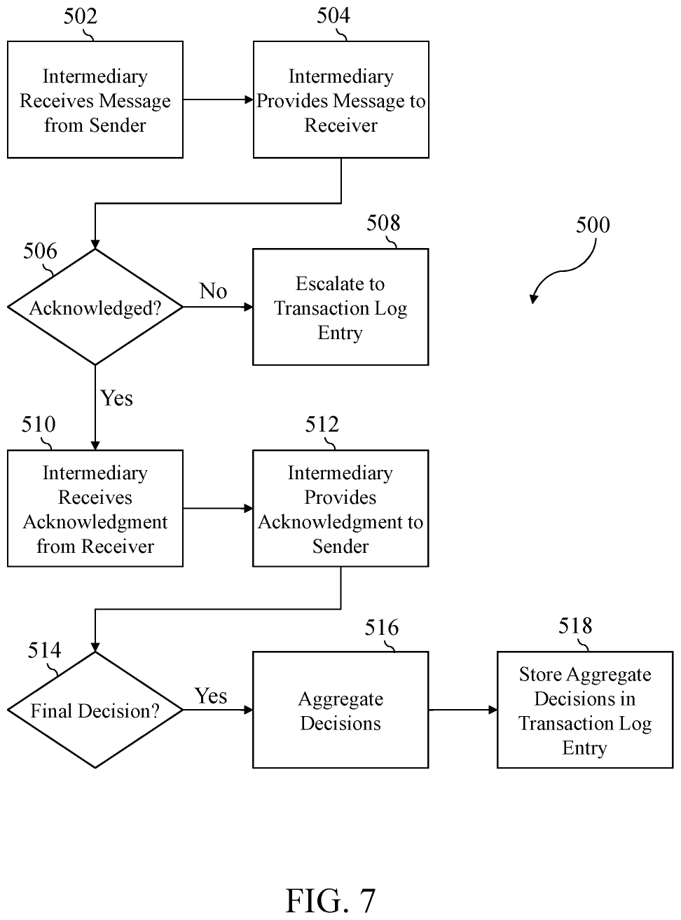

FIG. 7 is a flowchart of an exemplary set of steps (500) that could be performed to exchange a plurality of messages over an open transactional exchange channel. This exchange channel may also be referred to as a private transaction exchange channel, although the exchange channel may include both publicly viewable components (e.g., a public blockchain) and privately viewable components (e.g., messages privately exchanged between two parties), since the exchange of content occurs privately. As an example, the steps (500) may follow steps such as those described in the context of FIG. 6 and may be performed numerous times throughout the course of treatment or care of a particular patient. The intermediary (104) (e.g., via the messaging server (110)) may receive (502) a message (e.g., or may receive a plurality of messages) from a sender (e.g., either the payer (102) or the provider (106)), may store information from the message in file repositories or other data storage devices, and may provide (504) the message to the indicated recipient (e.g., either the provider (106) or the payer (102)). This may include transmitting the message to the recipient or notifying the recipient of the existence of the message and providing the recipient access to the message.

In each case that a message is provided (504) to a receiver, a response or acknowledgment is expected from the receiver that verifies that the message was received and verifies the content of the received message. When a message is provided to the receiver but not acknowledged (506) within a reasonable time, the non-response may be escalated (508) outside of the private messaging channel and logged to the transaction log (108) as a publicly viewable indication of a failed acknowledgment. Such escalation may be performed by the sender, the intermediary (104), or both, and it may serve as a publicly viewable indication of the unacknowledged message. Since each party to the transaction is monitoring the transaction log (108) using a service such as the transaction log monitor (204), the escalated request for acknowledgment should now be available to the recipient on both the private channel (e.g., provided (504) to receiver) and on the public channel (e.g., viewable on the transaction log (108)). In the event that acknowledgment did not occur for technical reasons, the escalated request for acknowledgment provides the receiver an additional opportunity to respond. In the event that the message was not acknowledged for business reasons (e.g., a failed business process responsible for addressing the message, or an intentional choice to not acknowledge), the publicly viewable escalated request creates an accountability record indicating that failure.

Where the provided (504) message is acknowledged (506), the intermediary (104) may receive (510) an acknowledgement message from the recipient and may provide (512) the acknowledgment to the original sender. Providing (512) the acknowledgment may include pushing the acknowledgment message to the sender, notifying the sender of the message and providing access to the message, or both. Each of the message and the acknowledgment may include unique, sequentially determined number or hash, as will be described in more detail below. As an example, the provided (504) message may include, in addition to its normal contents, a unique hash code built from a combination of prior hashes and its own message content, while the provided (512) acknowledgment may include a unique hash value built from a combination of the provided (504) message hash and its own acknowledgment contents (e.g., an acknowledgment message or status, a timestamp, a recipient identifier, etc.).

Messages may be exchanged as shown in FIG. 7 throughout the care of the patient, and upon completion of the care a party may transmit a message that indicates a final decision or outcome of the exchange. This may happen, for example, when the patient is discharged by the provider (106), and the payer (102) provides a final coverage decision or summary to the provider (106) indicating what portion of the cost of care the payer (102) will pay and what portion should be invoiced to the patient. When a message is received that indicates a final decision (514), one or more of the parties to the transaction may aggregate (516) data from prior exchanged messages to extract details that they wish to be publicly captured and preserved that are associated with the exchange.

This may include totaling, for each message in the exchange chain, the total cost of services provided, the total amount of insurance benefit provided, an amount of cost or requested services that were denied, a number of messages that were escalated before acknowledgment, the average amount of time between a message and an acknowledgment for each party, and other similar information. Such information that is aggregated (516) may then be added (518) to the transaction log (108) and associated with that instance of the transactional exchange channel. Since the transaction log (108) may be publicly viewable, aggregate information will typically not include any patient health information but will only contain aggregated and anonymized details. Such aggregate information may be useful for parties that are interested in evaluating individual parties to a transaction, which may include evaluating a particular provider (106) to determine how responsive they are, how many of their requested payments or services are denied, or other information related to the exchange of message. Such information may also be used to evaluate a payer (102) to determine how responsive they are, how often requests are publicly escalated, what percentage of requested payments or services they authorize or deny, and other information.

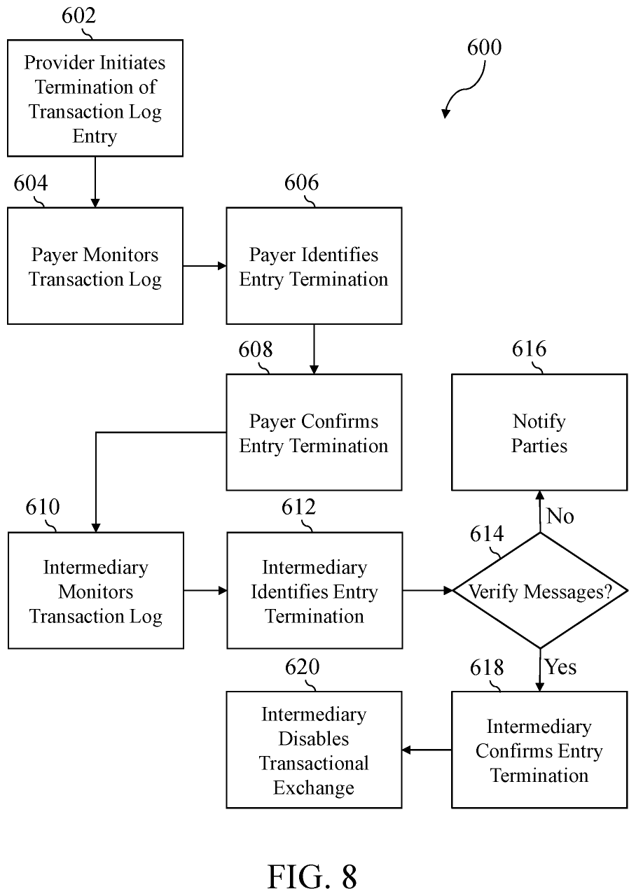

FIG. 8 is a flowchart of an exemplary set of steps that could be performed to close the transactional exchange channel upon completion of patient care or when the channel is otherwise unneeded. Such steps may be performed after a final decision (514) is received, or another message or indication is received that the channel may or should be closed. While any party may request that the transactional exchange channel be closed, often the provider (106) will initiate (602) termination of the transaction log chain that was previously opened by steps such as those in FIG. 6. Termination (602) of the transaction log chain, or exchange channel, may be initiated by writing information to a block, public ledger, or other database indicating that the party no longer needs the channel.

In the example where the provider (102) is the initiating party, the payer (102) monitors the transaction log (108) for changes associated with their unique identifier, or changes associated with a unique identifier for a patient or patient care that they are involved in and identifies (606) the initiated termination of the transaction log entry based on such information. Unless the payer (102) has a reason to maintain the channel (e.g., such as where the payer has not received an acknowledgment of a past message shared through the private channel, has received new information related to the patient or patient event from another source, or other scenarios), the payer (102) may confirm (608) termination of the transaction log chain or transactional exchange channel by creating a record on the blockchain, public ledger, or other database that indicates their agreement that the channel be finalized and closed.

The intermediary (104) may monitor (610) the transaction log for changes associated with their own unique identifier, or a unique identifier for a patient or patient care that they are associated with, and the intermediary (104) may identify (612) the initiated and confirmed termination of the transaction log entry or transactional exchange channel. The records indicating initiation of and confirmed termination of the transactional exchange channel may be referred to herein as a channel close record and a close confirmation record, which may include records identified on the transaction log (108) or other exchanged information requesting and confirming termination of the exchange channel. The intermediary (610) may validate (614) one or more messages in the chain of messages from the exchange to verify that they were each acknowledged and that hashes match the message content, stored files, or other information. Where the message chain cannot be verified (614), the intermediary (104) may notify (616) one or both of the parties and indicate the reason why the channel cannot yet be closed, to allow the parties to resolve the conflict (e.g., by acknowledging a previous message, providing missing information or files, or other actions). Where the message sequence can be verified (614), the intermediary may confirm (618) the closing of the transactional log entry by creating a record on the blockchain, public ledger, or other database indicating that the sequence of messages is valid and complete and that the channel is being closed. The intermediary (620) may then disable transactional exchanges of messaging over that private channel by de-authorizing the parties to communicate with each other over the channel, or requiring that a new channel be opened, or an old channel reopened, by following steps such as those described in FIG. 6.

One or more of the steps of initiating (602) termination, confirming (608) termination by the payer (102), and confirming (618) termination by the intermediary (104) may include providing data to the blockchain, public ledger, or database that completes the sequence of unique information that allows any message of the chain to be verified without revealing the contents of the entire chain. This may include recording the unique hash from the final message, final message acknowledgment, or both to the publicly viewable transaction log (108). In this manner, any transaction can be verified from any stage forward, starting at the opening message, which was recorded on the transaction log (108), through the private channel messages, and termination on the closing message, which was recorded on the transaction log (108), by recalculating the hashes at each step.

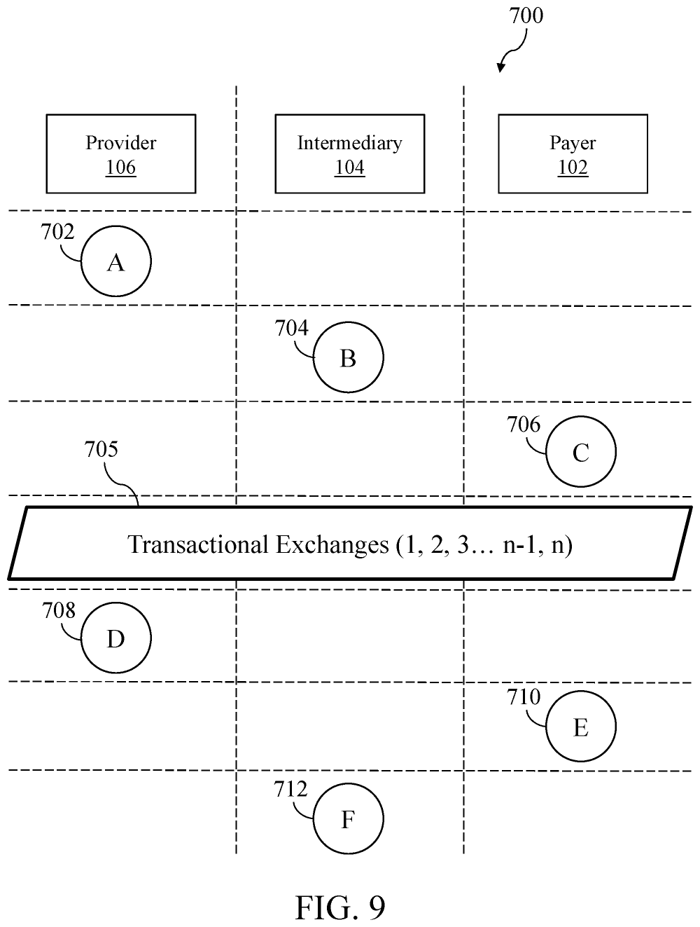

As an example of the above, FIGS. 9-11 provide several illustrations. FIG. 9 is a schematic diagram that illustrates aspects of FIGS. 5-8. Sequence chart (700) illustrates an exemplary transactional communication scenario, in sequence from top to bottom, from the opening of the transactional channel through the closing, with actions performed by each party being in the column underneath that party. While these steps are shown in sequence, it should be understood that in some implementations parties may perform actions in parallel with each other (e.g., the payer (102) and the intermediary (104) may simultaneously monitor for, identify, and confirm messages in the transactional exchange and requests that channels be opened or closed). With reference to FIG. 9, events represented by a circular object indicate records being written to (e.g., messages being shared through) the transaction log (108), such as described in connection with FIGS. 6 and 8, while events occurring within the non-rectangular parallelogram indicate messages being transmitted over a private channel, such as described in connection with FIG. 7.

Initially, the provider (106) may transmit a patient identifier, intermediary identifier, payer identifier, and exchange identifier to the intermediary (104) and create a transactional channel request (702) that includes the same exchange identifier on the transaction log (108). The intermediary (104) may see that it was identified in the transactional channel request (702) and, if it has received the data bundle with the exchange identifier that was indicated in the transactional channel request (702), create an intermediary confirmation (704) on the transaction log (108). The payer (106) may observe its identifier in the transactional channel request (702), note the intermediary confirmation (704) that indicates the initial information was received by the intermediary (104), and create a payer confirmation (706) on the transaction log (108). Alternatively, if payer (106) is identified in the transactional channel request (702) but does not have any affiliation with the patient identified in the transactional channel request (702), payer (106) may expressly reject the opening of the transactional channel by creating a payer non-confirmation entry (not shown) on the transaction log (108).

Next, after the payer confirmation (706) appears on the transaction log (108), a series of one or more transactional exchanges of messages (705) may be sent over the private channel. The transactional exchange of messages (705) may be mostly or entirely invisible to outside observers viewing only the transaction log (108), except in cases where, for example, aggregate decisions are added (518) to another repository such as the transaction log (108), or other hash or verification information is stored separately (e.g., such as where message hashes may be added to the transaction log (108) individually or in bulk from time-to-time, in order to aid in later verification of the sequence of hashes). When aggregate decisions are added (518) to the transaction log, or when records relating to individual or batches of messages are added to the transaction log, they may be referred to as a message record. Further, the combination of requests and confirmations (702, 704, 706) may be referred to as a transaction start record, which may include one or more requests, confirmations, or other records or information.

When the provider (106) determines that the channel is no longer needed, the provider (106) may create a transaction channel close request (708) on the transaction log (108). When the payer (106) identifies the transaction channel close request (708), the payer (106) may verify that it has completed use of the channel and create a transaction channel close confirmation (710) on the transaction log (108). When the intermediary (104) identifies the transaction channel close confirmation (710), the intermediary (104) creates a channel closed indicator (712) on the transaction log (108) and prevents further communication via the associated transactional exchange. When used herein, an exchange termination record should be understood to include the channel closed indicator (712) or other similar information that is created on the transaction log (108) or otherwise exchanged in order to indicate the termination of an exchange channel. Further, the combination of requests and confirmations (708, 710, 712) may be referred to as a transaction termination record, which may include one or more requests, confirmations, or other records or information.

While in some embodiments intermediary (104) is a public, governmental, or private entity organized and existing to store sensitive information in a health care or other information ecosystem, alternative embodiments of intermediary (104) comprise an automated system, such as software running on one or more separate processors or a smart contract established between the parties, to perform the tasks described herein as being performed by the intermediary (104). In some of these alternative embodiments, the smart contract exists on a private blockchain or other distributed ledger or database (such as the store that holds the messages discussed herein in connection with FIGS. 9-11) and has programmatic access to transaction log (108). In other embodiments, the smart contract exists on the same device and/or data structure as transaction log (108) and has programmatic access to messaging server (110), file server (112) and any other data it needs to automatically perform the tasks of intermediary (104). Other structures and organizational features will occur to those skilled in the art in view of this disclosure.

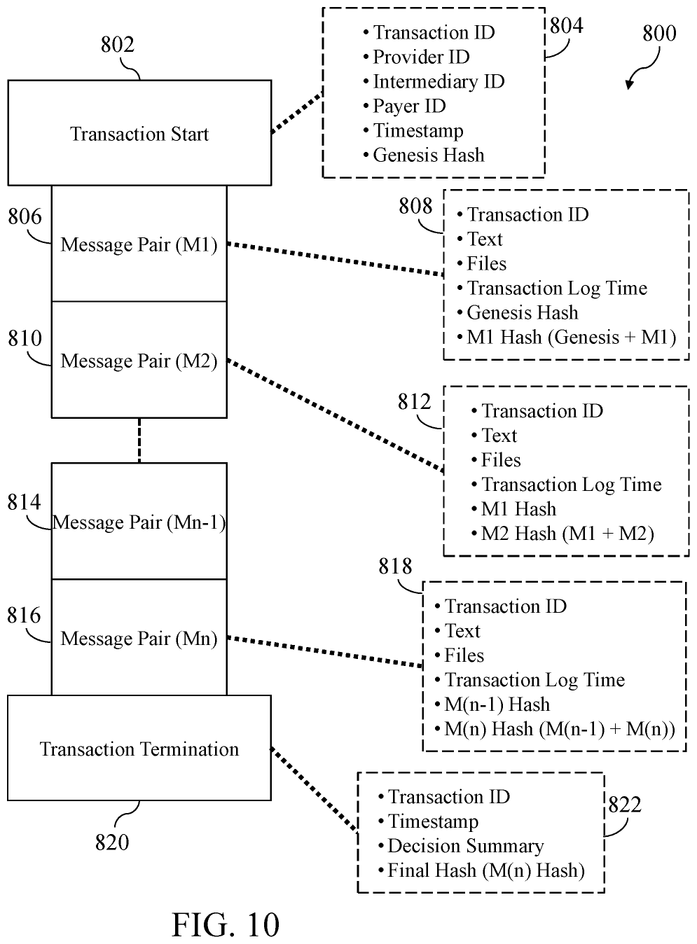

FIG. 10 is a schematic diagram that illustrates an exemplary transaction chain (800) that may be created by performing steps such as those in FIGS. 5-8, and as illustrated in FIG. 9. A transaction start (802) record written to the transaction log (108) may include publicly viewable records (702, 704, 706) from one or more parties to the exchange that individually or collectively contain a set of information (804) that may include, for example, a transaction ID, which may be a unique, anonymous identifier associated with a patient, a patient's care, or a particular transaction channel; a provider ID, which may uniquely identify a provider associated with the transaction; an intermediary ID, which may uniquely identify an intermediary associated with the transaction; a payer ID, which may uniquely identify a payer associated with the transaction; and a timestamp, which may indicate the date and time at which the transaction started, and which may be sourced from a computer system of a transacting party, or may be sourced from a publicly available universal time indicator (e.g., a world clock API, a timestamp or other data determinative of time that is associated with the records created on the transaction log (108), such as a block height).

The set of information (804) may also include a genesis hash, which serves as the starting hash, and that will be carried, in part, through the entire sequence of messages and then written to the transaction log (108) upon termination of the channel to enable verification of one or more messages along the chain. The genesis hash may be a unique hash created from input information, which may include one or more of the components of the set of information (804) itself, may include files, electronic messages, or other data sent (402) as initial information at the start of the transaction, and may include information from the transaction log (108) associated with the transaction start, such as a unique hash associated with a block in a blockchain, an entry in a public ledger, or a record in a database. Such information may be aggregated and hashed, or may be individually hashed, and then concatenated and re-hashed, in any order that is desired, so long as the method and order of hashing is known or ascertainable to allow for later verification of one or more messages.

A message pair, M1 (806) is a first message sent across the private channel after the transaction start (802). While M1 (806) is described as a message pair (e.g., a message sent, and an acknowledgement returned, as described above), M1 (806) could also be considered a sent message, and M2 (810) could be considered an acknowledgment of the sent message, with FIG. 10 being illustrative of the various embodiments of the disclosed technology in either case. A set of message information (808) for M1 (806) may include the transaction ID, text, which may include the body of a message, or a description of the message contents, files, which may include files of various types, formats, and content, such as x-ray images in various forms, invoices in various forms, patient data in various forms, and other data as will be apparent to one of ordinary skill in the art, and a transaction log time, which may be a universal time taken from the transaction log (108) or another source at the time of the message. M1 (806) also contains the genesis hash, carried over from the transaction start (802), as well as a unique hash for M1 (806). The M1 hash may be a hash code produced from inputs that may include any portion of the set of message information (808), as well as the genesis hash. Such information may be combined or concatenated together in a desired order and manner, so long as that manner is consistent and allows for future verification.

A message pair M2 (810) follows M1 (806) on the private channel, and a set of message information (812) includes data similar to that of M1 (806), except that the M1 hash is included, as well as an M2 hash, which itself may be a hash of the combination of the M1 hash and any or all of the contents of the message M2 (810). A number of messages may follow, including a penultimate message M(n−1) (814), and a final message M(n) (816). A set of message information (818) associated with final message M(n) (816) includes information similar to prior messages, with a hash from M(n−1) (814) and an M(n) hash created from the M(n−1) hash and information from M(n) (816), by applying a desired hash function to inputs according to a known selection and order.

A transaction termination (820) follows M(n) (816) in the sequence of messages, with M(n) (816) being the final message transmitted over the private channel, and the transaction termination (820) being created on the transaction log (108). A set of transaction termination information (822) may include the transaction ID and timestamp, as previously discussed, and may also include a final hash, which may be the hash from M(n) (816), to complete the sequence of hashed values, such that at least the genesis hash and the final hash are visible on the transaction log (108).

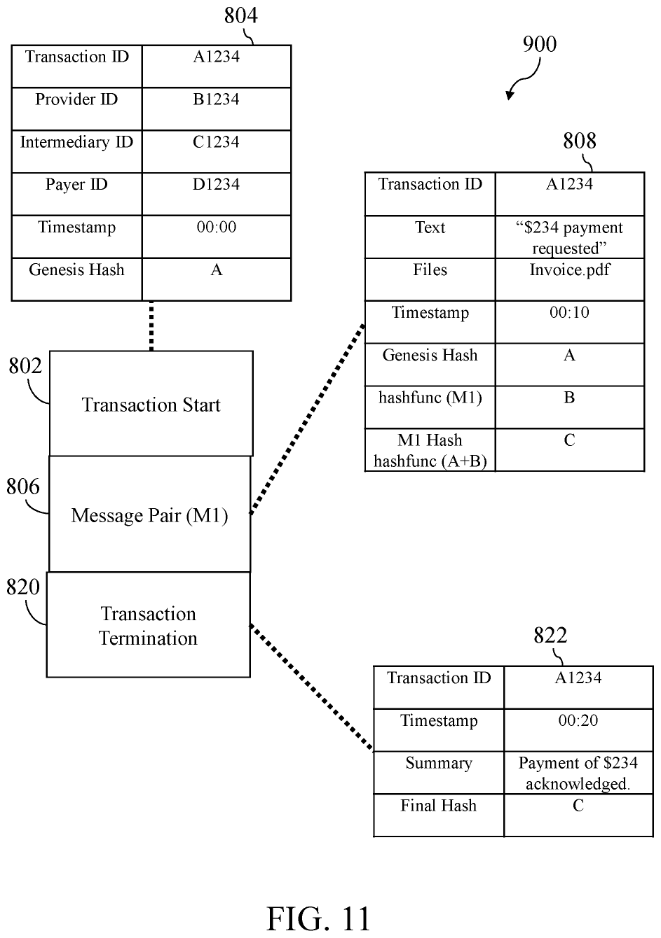

To provide further detail on the above example, FIG. 11 is a schematic diagram that illustrates data (900) associated with the exemplary transaction chain (800) of FIG. 10. As can be seen in that diagram, the set of information (804) includes various unique identifiers, a timestamp, and a genesis hash value, A. The set of message information (808) for M1 (806) includes the unique transaction ID shared between all messages in the transaction chain (800), text describing the message, a file associated with the message, and a timestamp showing when the message was transmitted. Also included in the message information (808) for M1 (806) are the genesis hash, A, a local hash for M1 (806), the value B, that is determined by executing a hashing function “hashfunc” taking the previous content of M1 (806) as input, and an M1 hash, provided by executing hashfunc with inputs of A+B to produce C. In this example, M1 (806) is the first and final message sent through the private channel, so the next message is the transaction termination (820). The set of transaction termination information (822) includes the same transaction ID as had been used throughout the transaction chain (800), a new timestamp, a summary (e.g., such as the aggregate decisions (516) added (518) with or after the final message), and the final hash C, from the final message, M1 (806).

When used herein, a hash value should be understood to include one or more of an integer string that identifies input data, a set of encoded data or information that represents a digest of its input values (where it is generally computationally infeasible to reverse the process), or other similar data. Further, a hash value that is associated with (e.g., contained within or linked to) a record or entry in the transaction log (108) or another database may be described in relation to that record or entry. As an example, an open request hash value may be a hash value associated with an open request record, a confirmation hash value may be associated with an open confirmation record, a response confirmation hash value may be associated with a response confirmation record, a public message hash value may be associated with a message record, a prior message hash value may be associated with a message record that has previously been written to the transaction log (108), a channel close hash value may be associated with a channel close record, a final hash value may be associated with an exchange termination record, a current message hash value may be associated with a message record that is currently being written to the transaction log (108), and a plurality of hash values may be understood to include any combination of the above hash values.

It can be seen from FIG. 11 that the successful transmission, acknowledgment, and contents of the message M1 (806) can be verified by a party having access to the transaction start (802), which is available via the transaction log (108), the local hash B of the message M1 (806), with the text, files, and other information being unnecessary, and the final hash C, available via the transaction log (108). Thus, for a party wishing to authenticate, audit, or otherwise review any individual message of the transaction chain (800), the only private information needed is the prior message hash (e.g., the genesis hash in the case of FIG. 11), and the current message hash, or the local hash of the message to be authenticated and any subsequent messages prior to the transaction termination (820). Where such information may be provided by any party to the transaction or logged to the transaction log (108) to make it publicly available, the associated message may be verified, including its content, time of receipt, acknowledgment, and other details, by recalculating the hash codes until the final hash is reached, and verifying that the final hash matches the transaction log (108).

It may be noted by those skilled in the art that hash functions suitable for producing any of the hashes described herein will, in various embodiments, be keyed, salted, both, or neither. In various embodiments, the hashing operations used to create the various hash values discussed herein will have different ones of these features, while in other embodiments such operations will be consistently applied.

While the above examples are disclosed in the context of health care and health insurance, it should be understood that one or more aspects of the transactional data exchange may be implemented in various contexts in order to provide robust and verifiable exchange of data as described above. As an example, one or more of the aspects, features, advantages, or steps described herein may also apply to various financial transactions, including loan applications, real estate purchases, purchases of goods and services, and other contexts.

It should be understood that any one or more of the teachings, expressions, embodiments, examples, etc. described herein may be combined with any one or more of the other teachings, expressions, embodiments, examples, etc. that are described herein. The teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

What is claimed is:

1. A messaging system for communications among a plurality of participants comprising a first participant, a second participant, and a third participant, the messaging system comprising a messaging server, a public database, and one or more processors configured to:
in response to a channel open request from the first participant, create an open request record in the public database that corresponds to the channel open request;
create an open confirmation record in the public database that corresponds to the open request record, where the open confirmation record comprises a response identifier that is associated with the second participant;
identify a response confirmation record in the public database that corresponds to the open confirmation record, where the response confirmation record originated from the second participant;
in response to the identification of the response confirmation record, enable a private transactional exchange channel configured to allow the first participant and the second participant to exchange information;
process a plurality of messages from a sender to a recipient via the private transactional exchange channel, the sender and the recipient for each message being selected from the plurality of participants, the processing comprising, for each message:
provide the message to the recipient;
receive an acknowledgment from the recipient;
provide the acknowledgment to the sender; and
create a message record in the public database based on the message and the acknowledgment.

2. The messaging system of claim 1, wherein the public database is a distributed ledger.

3. The messaging system of claim 1, wherein the public database is a blockchain.

4. The messaging system of claim 1, wherein the one or more processors are further configured to:
identify a channel close record and a close confirmation record in the public database that each correspond to the private transactional exchange channel; and
in response to identification of the close confirmation record, disable the private transactional exchange channel and create an exchange termination record in the public database that corresponds to the close confirmation record.

5. The messaging system of claim 4, wherein
the one or more processors are further configured to verify a channel close hash value of the channel close record,
the exchange termination record includes a final hash value that is based on the channel close hash value, and
the channel close hash value is configured to allow an audit process to verify each of a plurality of hash values, in sequence, including the open request hash value.

6. The messaging system of claim 5, wherein the plurality of messages comprises a first message and one or more subsequent messages, and each subsequent message includes:
a transaction identifier that identifies the private transactional exchange channel;
a message body;
a prior message hash value;
a current message hash value that is based on the transaction identifier, the message body, and the prior message hash value; and
wherein the public message hash value is based on the prior message hash value and the current message hash value.

7. The messaging system of claim 5, further comprising an audit server configured to:
receive an audit request comprising:
identification of an audited message record in the public database;
an audited message that is associated with the audited message record;
the prior message hash value that is associated with the audited message record; and
the public message hash value that is associated with the audited message record;
recalculate, based on the prior message hash value and the public message hash value, a plurality of hash values, in sequence, starting from the audited message record and ending at a close confirmation record; and
where a recalculated final hash value matches a final hash value that is associated with the close confirmation record, provide an indication that the content of the audited message is accurate.

8. The messaging system of claim 7, wherein the content of the audited message comprises:
a message body;
a time of receipt; and
a transaction identifier.

9. The messaging system of claim 1, wherein the public database is configured so that each record created in the public database is immutable.

10. The messaging system of claim 9, wherein the public database comprises a blockchain database.

11. The messaging system of claim 10, wherein the configurations of at least one of the one or more processors are comprised of a plurality of smart contracts that are stored in the blockchain database.

12. The messaging system of claim 1, wherein the one or more processors are further configured to:
verify an open request hash value from the open request record;
create the open confirmation record to include a confirmation hash value that is based on the open request hash value;
verify a response confirmation hash value of the response confirmation record; and
create each message record to include a public message hash value that is based on:
the response confirmation hash value, when that message is a first message of the plurality of messages; and
a prior message hash value, when that message is not the first message of the plurality of messages.

13. The messaging system of claim 1, wherein the messaging server is administrated by a health information exchange entity, the first participant is a health service provider, and the second participant is a health service payment provider.

14. The messaging system of claim 13, wherein the health information exchange entity and the health service provider are not legally related entities.

15. The messaging system of claim 1, further comprising a first participant server that is associated with the first participant, wherein the first participant server is configured to:
send the channel open request to the messaging server and create the open request record in the public database;
send or receive the plurality of messages via the private transactional exchange channel; and
create one or more of the channel close record and the close confirmation record in the public database.

16. The messaging system of claim 15, wherein the first participant server is further configured to:
communicate with the private transactional exchange channel via an exchange interface;
identify an electronic message that is associated with the enabled private transactional exchange channel, but is received via a channel other than the private transactional exchange channel; and
convert and transmit the electronic message to the messaging server via the private transactional exchange channel.

17. A method for managing communications among a plurality of participants comprising a first participant, a second participant, and a third participant, using a messaging system comprising a messaging server, a public database, and one or more processors, the method comprising:
in response to a channel open request from the first participant, creating an open request record in the public database that corresponds to the channel open request;
creating an open confirmation record in the public database that corresponds to the open request record, where the open confirmation record comprises a response identifier that is associated with the second participant;
identifying a response confirmation record in the public database that corresponds to the open confirmation record, where the response confirmation record originated from the second participant;
in response to the identification of the response confirmation record, enabling a private transactional exchange channel configured to allow the first participant and the second participant to exchange information;
processing a plurality of messages from a sender to a recipient via the private transactional exchange channel, the sender and the recipient for each message being selected from the plurality of participants, the processing comprising, for each message:
providing the message to the recipient;
receiving an acknowledgment from the recipient;
providing the acknowledgment to the sender; and
creating a message record in the public database based on the message and the acknowledgment.

18. The method of claim 17, further comprising:
identifying a channel close record and a close confirmation record in the public database that each correspond to the private transactional exchange channel;
in response to identification of the close confirmation record, disabling the private transactional exchange channel and create an exchange termination record in the public database that corresponds to the close confirmation record; and verifying a channel close hash value of the channel close record, wherein:
the exchange termination record includes a final hash value that is based on the channel close hash value, and
the channel close hash value is configured to allow an audit process to verify each of a plurality of hash values, in sequence, including the open request hash value.

19. The method of claim 18, wherein the plurality of messages comprises a first message and one or more subsequent messages, and each subsequent message includes:
a transaction identifier that identifies the private transactional exchange channel;
a message body;
a prior message hash value;
a current message hash value that is based on the transaction identifier, the message body, and the prior message hash value; and
wherein the public message hash value is based on the prior message hash value and the current message hash value.

20. A messaging system comprising a messaging server, a public database, and one or more processors configured to:
create a transaction start record in the public database in response to requests from a first participant and a second participant, wherein the transaction start record comprises one or more records that include a transaction identifier and a genesis hash, where the genesis hash is based on data received from the first participant;
enable a private transactional exchange channel between the first participant and the second participant based on the transaction start record;
receive a plurality of messages via the private transactional exchange channel, where each message in the plurality of messages is from a sender to a recipient, and for each message:
provide that message to the recipient;
receive an acknowledgment from the recipient;
provide the acknowledgment to the sender; and
create a message record in the public database that includes the transaction identifier and a message hash based on the genesis hash, the message, and the acknowledgment; and
create a transaction termination record in the public database in response to requests from the first participant and the second participant, wherein the transaction termination record comprises one or more records that include the transaction identifier and a final hash that is based on the genesis hash and a plurality of message hashes that are associated with the transaction identifier.
